(19) United States
(12) Patent Application Publication
(10) Pub. No.: US 2002/0197341 A1
(43) Pub. Date: Dec. 26, 2002

(54) Physiologically synergistic mixtures of pomegranate extracts and methods of use thereof

(75) Inventor: Ephraim Philip Lansky, Haifa (IL)

Correspondence Address: DR. MARK FRIEDMAN LTD., C/o Bill Polkinghorn, Discovery Dispatch, 9003 Florin Way, Upper Marlboro, MD, 20772, US

(73) Assignee: RIMONEST LTD.

(21) Appl. No.: 10/217,430

(22) Filed: Aug. 14, 2002

Related U.S. Application Data
(63) Continuation-in-part of application No. 09/859,431, filed on May 18, 2001.
Application No. 09/859,431 is a continuation-in-part of application No. PCT/IL00/00800, filed on Nov. 29, 2000.
(60) Provisional application No. 60/167,694, filed on Nov. 29, 1999.

(51) Int. Cl. A61K 35/78

(52) U.S. Cl. 424/769; 424/776

(57) ABSTRACT

Physiologically synergistic mixtures of pomegranate extracts and methods of use thereof. Physiologically synergistic mixtures of pomegranate extracts are provided. A first mixture includes a pomegranate juice extract; and pomegranate seed oil. A second mixture includes pomegranate pericarp extract; and pomegranate seed oil. A third mixture a pomegranate pericarp extract; and a pomegranate juice extract. Disclosed methods inclod preparing components of disclosed mixtures, preparing the mixtures from the components and administering the resultant mixture to a subject in a physiologically effective amount so that a progression of a cell proliferation disorder is retarded.

This application is a CIP of U.S. application Ser. No. 09/859,431 filed May 15, 2001 which was a CIP of PCT application IL00/00800 filed Oct. 6, 2000, which claimed priority from U.S. Patent Application No. 60/167,694 filed Nov. 29, 1999.

### FIELD AND BACKGROUND OF THE INVENTION

The present invention relates to physiologically synergistic mixtures of pomegranate extracts useful in improving health and methods of use thereof. Specifically, the present invention relates to a mixture of pomegranate seed oil and a pomegranate juice extract or pericarp extract and to a mixture of pomegranate juice extract and pericarp extract. The invention further includes methods for treating cell proliferation disorders including but not limited to, cancer and benign prostatic hyperpla Mixtures of the present invention are expected to find utility in cosmetics, pharmaceutical compositions and articles of manufacture containing same.

Pomegranate (Punica granatum) has long been recognized as a fruit with many benefits for health.¹ The plant is botanically unique, having actually only one true botanical relative, the pomegranate precursor, Punica protopunica, restricted to the isolated island Socotra off the coast of Yemen. Corresponding to this botanical uniqueness is a parallel distinctiveness in terms of biochemistry. For example, pomegranate has long been recognized as the richest plant source of the female steroid hormone estrone,² and recently, the male hormone testosterone and another female steroid, estriol, have also been discovered in pomegranate seed oil.³ A wide range of polyphenolic compounds including flavonoids, anthocyanins and tannins have been characterized both in pomegranate juice⁴ and pericarp.⁵ Further, concentrations of these polyphenols extracted both from the fermented juice and the oil have been shown to be potently antioxidant in vitro and to additionally inhibit the eicosanoid enzyme lipoxygenase, and in the case of the polyphenols extracted from pomegranate seed oil, to also be significantly inhibitory of another eicosanoid pathway enzyme, cyclooxygenase.⁶ However, previous research into medical applications of pomegranate products has focused on isolation and purification of single compounds or extracts. Thus, the potential physiologic synergy between various portions of the pomegranate fruit has been ignored. ¹ Frawley, D and Lad, V. The Yoga of Herbs: An Ayurvedic Guide to Herbal Medicine, Lotus Press, Twin Lakes, Wis. 1986. ² Moneam, N. M. A., El Sharaky, A. S., and Badreldin, M. M. Oestrogen content of pomegranate seeds. Journal of Chromotography 438: 438-442, 1988. ³ Abd El Wahab, S. M., El Fiki, S. F., Mostafa, S. F. and Hassan, A. E. B. Characterization of certain steroid hormones in Punica granatumL. seeds. Bulletin of the Faculty of Pharmacy of Cairo University 36(1): 11-15, 1998. ⁴ Artik, N., Cemeroglu, B., Burakami, H., and Mori, T. Determination of phenolic compounds in pomegranate juice by HPLC. Fruit Process 8 (12): 492-499, 1998. ⁵ Ben Nasr, C., Ayed, N., and Metche, M. Quantitative determination of the polyphenolic content of pomegranate peel. Z Lebensm Unters Forsch 203 (4): 374-378, 1996. ⁶ Schubert, S. Y., Lansky, E. P., and Neeman, I. Antioxidant and eicosanoid enzyme inhibition properties of pomegranate seed oil and fermented juice flavonoids. Journal of Ethnopharmacology 66 (1): 11-17, 1999.

There is thus a widely recognized need for, and it would be highly advantageous to have, physiologically synergistic mixtures of pomegranate extracts useful in improving health and methods of use thereof as well as pharmaceutical compositions and articles of manufacture containing same.

### SUMMARY OF THE INVENTION

According to one aspect of the present invention there is provided a physiologically synergistic mixture of pomegranate extracts. The mixture includes: (a) a pomegranate juice extract; and (b) pomegranate seed oil.

According to another aspect of the present invention there is provided a physiologically synergistic mixture of pomegranate extracts. The mixture includes: (a) a pomegranate pericarp extract; and (b) pomegranate seed oil.

According to an additional aspect of the present invention there is provided a physiologically synergistic mixture of pomegranate extracts. The mixture includes: (a) a pomegranate pericarp extract; and (b) a pomegranate juice extract.

According to yet another aspect of the present invention there is provided a method of treating a cell proliferation disorder. The method includes: (a) preparing an extract of pomegranate pericarp; (b) mixing the extract with a quantity of pomegranate seed oil to form a mixture; (c) administering the mixture to a subject in a physiologically effective amount so that a progression of a cell proliferation disorder is retarded.

According to still another aspect of the present invention there is provided a method of treating a cell proliferation disorder. The method includes:(a) preparing an extract of pomegranate juice; (b) mixing the extract with a quantity of pomegranate seed oil to form a mixture; (c) administering the mixture to a subject in a physiologically effective amount so that a progression of a cell proliferation disorder is retarded.

According to yet an additional aspect of the present invention there is provided a method of treating a cell proliferation disorder. The method includes: (a) preparing a first extract of pomegranate pericarp; (b) preparing a second extract of pomegranate juice; (c) mixing the first and second extracts to form a mixture; (d) administering the mixture to a subject in a physiologically effective amount so that a progression of a cell proliferation disorder is retarded.

According to further features in preferred embodiments of the invention described below, the mixture further includes a pomegranate pericarp extract.

According to still further features in the described preferred embodiments the juice extract includes an extract prepared using a solvent selected from the group consisting of water, an alcohol, ethyl acetate and carbon

According to still further features in the described preferred embodiments the mixture further includes: a pomegranate juice extract.

According to still further features in the described preferred embodiments the pericarp extract includes an extract prepared using a solvent selected from the group consisting of water, an alcohol, ethyl acetate and carbon dioxide.

According to still further features in the described preferred embodiments the progression is retarded prior to appearance of clinical symptoms so that the method constitutes a prophylactic treatment for a cell proliferation disorder.

According to still further features in the described preferred embodiments the method further includes preparing an additional extract of pomegranate juice and further including the additional extract in the mixture.

According to still further features in the described preferred embodiments the method further includes preparing an additional extract of pomegranate pericarp and further including the additional extract in the mixture.

The present invention successfully addresses the shortcomings of the presently known configurations by providing physiologically synergistic mixtures of pomegranate extracts useful in improving health and methods of use thereof. Further, the present invention directly contradicts prior art configurations by re-combining pomegranate products after their separatiuon or purification in order to increase their physiologic potency in a synergistic fashion.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a flow diagram showing production steps in manufacture of 1000 doses of an elixir for women according to the present invention;

FIG. 2 is a flow diagram showing production steps in manufacture of 1000 doses of an elixir for men according to the present invention;

FIG. 3 is a graph illustrating the effect of pomegranate juice extract (w), pomegranate seed oil, and combinations thereof on growth of D-145 prostate cancer cells in culture;

FIG. 4 is a histogram illustrating the effect of pomegranate juice extract (w), pericarp extract (P), pomegranate seed oil, and combinations thereof on the ability of PC-3 prostate cancer cells to penetrate a Matrigel membrane in culture;

FIG. 5 is a histogram illustrating the effect of pomegranate juice extract (w), pericarp extract (P), and combinations thereof on growth of D-145 prostate cancer cells in culture.

### DESCRIPTION OF THE PREFERRED EMBODIMENTS

The present invention is of physiologically synergistic mixtures of pomegranate extracts and/or pomegranate seed oil. The invention is further of methods of treating cell proliferation disorders which include administration of these physiologically synergistic mixtures to a subject. The invention is expected to find utility in cosmetics as well as in prophylaxis or treatment of a variety of medical conditions including, but not limited to, cancer, especially hormone dependent cancer.

The principles and use of a mixture physiologically synergistic mixtures of pomegranate extracts and/or pomegranate seed oil same according to the present invention may be better understood with reference to the drawings and accompanying descriptions.

For purposes of this specification and the accompanying claims, the terms “pericarp”, “rind” and “peel” are considered synonymous and are used interchangeably.

For purposes of this specification and the accompanying claims, the terms “pericarp extract”, includes pomegranate peel residue present in pomegranate juice as a result of a juicing process, an aqueous extract of pomegranate peel, an alcohol extract of pomegranate peel, an extract performed with an organic solvent which is not an alcohol, and a supercritical fluid extract of pomegranate peel. The supercritical fluid extract may be conducted with, for example CO₂, ethanol, methanol, water, or combinations thereof Pericarp extract is known to contain quercetin, kaempferol, luteolin, derivatives thereof and combinations thereof such that these compounds must be included within the definition of “pericarp extract”.

For purposes of this specification and the accompanying claims, the terms “alcohol extract”, includes extracts prepared using methanol, ethanol, propanol, butanol and l/or longer chain alcohols and/or combinations thereof.

For purposes of this specification and the accompanying claims, the term “pomegranate seed oil” includes the result of a process such as, for example, expeller pressing, supercritical fluid extraction with carbon dioxide, and/or lyophilization.

For purposes of this specification and the accompanying claims, the term “pomegranate juice” refers to unprocessed pomegranate juice, fermented pomegranate juice, dried pomegranate juice, dried fermented pomegranate juice, partially fermented pomegranate juice, partially dried pomegranate juice, partially fermented partially dried pomegranate juice, reduced pomegranate juice, partially reduced pomegranate juice and lyophylysates thereof. For purposes of this specification and the accompanying claims, the term pomegranate juice extract includes, but is not limited to, supercritical fluid extracts such as, for example supercritical fluid extracts prepared with CO₂, water, ethanol, methanol and all other chemical solvents and combinations thereof.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present invention is of physiologically synergistic mixture of pomegranate products. The ability of mixtures according to the present invention to inhibit cancer stems from their ability to inhibit cellular proliferation and prevent invasion as detailed hereinbelow in the examples section.

Thus, the present invention is embodied by a physiologically synergistic mixture of pomegranate extracts including a pomegranate juice extract and pomegranate seed oil. Preferably, the mixture further includes a pomegranate pericarp extract. The juice extract may includes, for example, an extract prepared using a solvent such as water, an alcohol, ethyl acetate or carbon dioxide.

The present invention is further embodied by a physiologically synergistic mixture of pomegranate extracts including a pomegranate pericarp extract and pomegranate seed oil. Preferably, the mixture further includes a pomegranate juice extract. The pericarp extract may include, for example, an extract prepared using a solvent such as water, an alcohol, ethyl acetate and carbon dioxide.

The present invention is further embodied by a physiologically synergistic mixture of pomegranate extracts including a pomegranate pericarp extract and a pomegranate juice extract.

According to the present invention., the physiologically synergistic mixtures of pomegranate extracts may be employed to treat, for example, cell proliferation disorders, such as cancer or benign prostatic hypertrophy.

Thus, the present invention is further embodied by a method of treating a cell proliferation disorder. The method includes preparing an extract of pomegranate pericarp. The method further includes mixing the extract with a quantity of pomegranate seed oil to form a mixture. The method further includes administering the mixture to a subject in a physiologically effective amount so that a progression of a cell proliferation disorder is retarded.

According to additional preferred embodiments of the invention, the method further includes preparing an additional extract of pomegranate juice and further including the additional extract in the mixture.

The present invention is further embodied by an additional method of treating a cell proliferation disorder. The method includes preparing an extract of pomegranate juice. The method further includes mixing the extract with a quantity of pomegranate seed oil to form a mixture. The method further includes administering the mixture to a subject in a physiologically effective amount so that a progression of a cell proliferation disorder is retarded.

According to additional preferred embodiments of the invention, the method further includes preparing an additional extract of pomegranate pericarp and further including the additional extract in the mixture.

The present invention is further embodied by another additional method of treating a cell proliferation disorder. The method includes preparing a first extract of pomegranate pericarp. The method further includes preparing a second extract of pomegranate juice. The method further includes mixing the first and second extracts to form a mixture. The method further includes administering the mixture to a subject in a physiologically effective amount so that a progression of a cell proliferation disorder is retarded.

According to the various methods of the invention, administering the mixture to a subject is preferably conducted prophylactically so that progression of the cell proliferation disorder is retarded prior to appearance of clinical symptoms. Thus, practice of the method constitutes a prophylactic treatment.

According to various preferred embodiments of the invention, pomegranate pericarp extract, juice extract or seed oil may make up a majority of the mixture.

Optionally, but also preferably, pomegranate seed cake extracts may be included in mixtures according to the present invention.

The pericarp extracts employed in the mixtures of the present invention preferably include an extract prepared using a solvent such as water, an alcohol, ethyl acetate or carbon dioxide. The seed cake extracts employed in the mixtures of the present invention include an extract prepared using a solvent selected from the group consisting of water, an alcohol, ethyl acetate and carbon dioxide.

Although alcohol extraction may be with methanol, ethanol, propanol (n- or iso-), or longer chain alcohols, eythanol is preferably employed because it is least toxic to human subjects.

According to preferred embodiments of the invention, solvent extraction is performed at supercritical conditions.

According to preferred embodiments of the invention, the mixtures further include emulsifiers such as lecithins or waxes, either natural or artificial.

As an illustrative, non-limiting, example of the invention, a claimed mixture may be further combined with additional food ingredients which confer the appearance of a recognizable food upon the product so that the result is a palatable food product such as chocolate, ice cream, an energy bar, a pasta, a cake, a pastry, a cookie or a candy.

As an additional illustrative, non-limiting, example of the invention, claimed mixture can be incorporated into cosmetics such as creams, rouges and face powders. Such cosmetics would be expected to exert an estrogenic, eicosanoid enzyme inhibitory, antioxidant, pro-apoptotic or related property to achieve an overall anti-aging effect.

In order to maximize the physiologic effect of mixtures according to the present invention juice extracts are preferably prepared from partially fermented or fully fermented juice.

Mixtures according to the present invention will preferably be provided as pharmaceutical compositions.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the mixture of pomegranate extracts accountable for the biological or physiologic effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma levels of the active ingredient sufficient to achieve the desired effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved. In prophylactic treatment, administration of doses is generally continued over a prolonged period.

The amount of a composition to be administered will, of course, be dependent on the subject being treated, the severity of the affliction, the manner of administration, the judgment of the prescribing physician, etc.

Pharmaceutical compositions may be further incorporated into an article of manufacture including instructions for use.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Mixtures according to the present invention are expected to find utility in treatment or prevention of cancers, including skin cancer, prostate cancer, breast cancer and colon cancer as well as in treatment or prevention of benign prostatic hypertrophy.

Mixtures according to the present invention may easily be provided in a variety of forms including, but not limited to, a liquid, a powder, granules, a tablet, a capsule, a gel-cap, an ointment, a lotion, a bath gel, a cream, a chewing gum, a food, a candy, an emulsion or a suppository.

Extracts used in preparing mixtures according to the present invention are preferably prepared from Wonderful cultivar pomegranates. More preferably, these pomegranates are organically grown, still more preferably, they are grown at Kibbutz Sde Eliahu in Israel.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

General references to standard laboratory techniques are provided throughout this document. The procedures therein are believed to be well known in the art and are provided for the convenience of the reader. All the information contained therein is incorporated herein by reference.

Materials and Methods:

Reference is now made to the following materials and methods which are employed in the examples detailed hereinbelow.

Bioluminescent ATP assay for cell proliferation: Dose responses of all three agents (P,W and Oil) were assessed in DU-145 cells with a proliferation assay using bioluminescence to measure cellular ATP. The cell proliferation assay (ViaLight HS, LumiTech, Nottingham, UK) was used according to manufacturer's instructions and with previously optimised conditions to ensure exponential proliferation of controls throughout the time course of the experiment Albrecht et al.(2002) “Pomegranate extracts potently inhibit growth, suppress invasion and modulate gene targets in prostate cancer cells.” Submitted for publication in Oncogene). Briefly, cells were plated into 96 well, white walled, tissue culture treated plates (2×10³ cells/well; Fisher Scientific Ltd. Loughborough, UK). The cells were exposed to treatments consisting of varying concentrations of P, W or Oil, either alone or in combination as indicated. The final volume per well was 100μl including media. Incubation was for 96 hr, with an additional aliquot of test extract(s) added at 48 hr. At the end of 96 hours, 100μl of nucleotide releasing reagent was added and the cells were incubated for 30 minutes at room temperature for extraction of the ATP. Liberated ATP was measured using20μl ATP monitoring reagent (containing luciferin and luciferase) and a micro plate luminometer (Berthold Detection Systems, Fisher Scientific Ltd.).

Cell invasion through Matrigel: A known in vitro invasion assay was employed. (Jiang et al. (1995) Cancer Research 55, 5043-5048,). Briefly, transwell chambers (Costar, Cambridge, Mass., USA) equipped with 6.5 mm diameter polycarbonate membrane (pore size 8μm) were precoated with a solublized tissue basement membrane (Matrigel 50μg/membrane; Collaborative Biochemical; Bedford, Mass.0;). After gel rehydration, 5×10⁴ prostate cancer cells were added to each membrane, with hepatocyte growth factor/scatter factor (HGF 40 ng/ml; graciously provided by Dr. T. Nakamura, Osaka, Japan) to induce invasion. Medium was added to the upper chamber of the insert with or without W, P or Oil, or combinations thereof. In all cases, the final concentration of test compound was 3 μg/ml. After 72 hours of incubation, the non-invasive cells in the upper chamber were carefully removed with a cotton swab, and cells which had migrated through the membrane and stuck to the lower surface were fixed and stained with crystal violet. The number of cells was quantified using an inverted microscope and was expressed as number of cells/high power field.

Statistical analysis. The interactions of two compounds were assessed by measuring the mean of P, W or Oil, acting alone or in combination. The mean observed combined effect was compared to the individual effects of the agents added together, using the Student's t-test (Rashid et al. (2002) Oncogene 20: 1860-72). Classification of the effects as synergistic was made when an experimental value was significantly greater than the predicted value.

Preparation of pomegranate polyphenol fractions was according to the method described by Schubert et al. (Schubert et al (1999) Journal of Ethnopharmacology 66 (1): 11-17,). For the fermented juice and aqueous extract of the rinds, the original liquids were combined with two times their volume of ethyl acetate, shaken vigorously, and left for 8 hours. The ethyl acetate phase was then dried in the vacuum evaporator at 40 degrees centigrade, and polyphenols resuspended in methanol. It is important to note that juice extract (W) often contains pericarp (P) as a result of the juicing process (Gil et al. 92000) J Agric Food Chem. 48:4581-9).

### Example 1

### Production of an Elixir from a Mixture of Pomegranate Products.

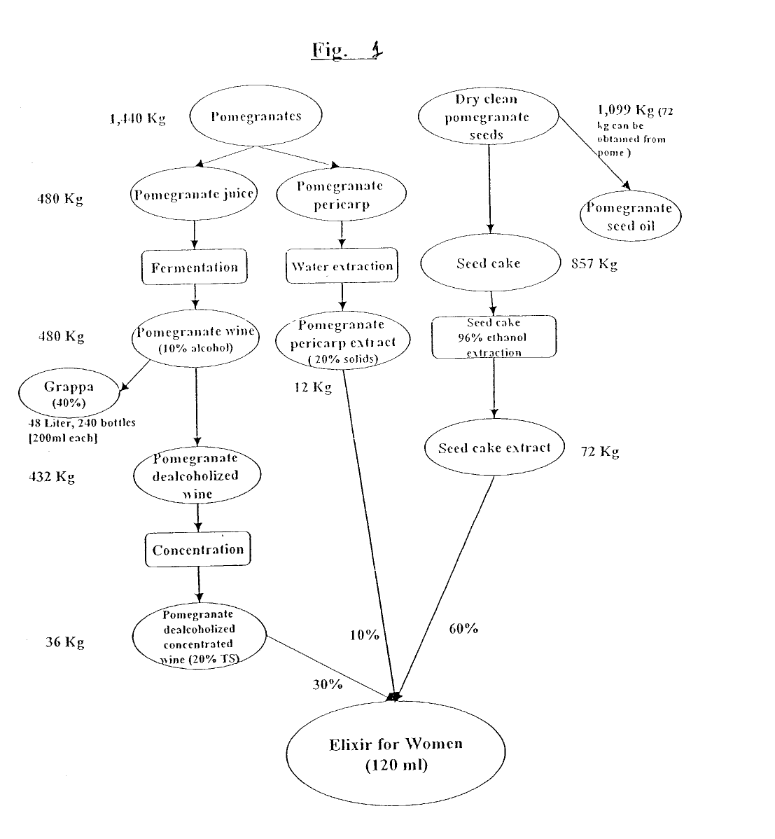

FIG. 1 shows production steps in manufacture of a pharmaceutical composition including 30% dealcoholized concentrated pomegranate wine, 10% aqueous extract of pomegranate pericarp, and 60% seed cake extract. The 120 ml of elixir represents 1000 doses of an elixir for women which could be delivered, for example as gel-caps. The elixir is expected to have beneficial effects in climacteria as well as to offer protection against development of breast cancer and to be beneficial in treating breast cancer. Raw materials are 1440 Kg of whole pomegranates and 1099 kg of pomegranate seeds. The pomegranates are initially processed into juice and pericarp. The juice is then fermented and distilled. The dealcoholized wine is then reduced to a 36 kg. Concentrate containing approximately 20% total solids. The pericarp is subjected to an aqueous extraction which produces a pericarp extract containing approximately 20% total solids. The seeds are “dry cleaned” or solvent extracted to produce a seed cake which is further extracted with ethanol. The resultant seed cake extract becomes a component of the elixir.

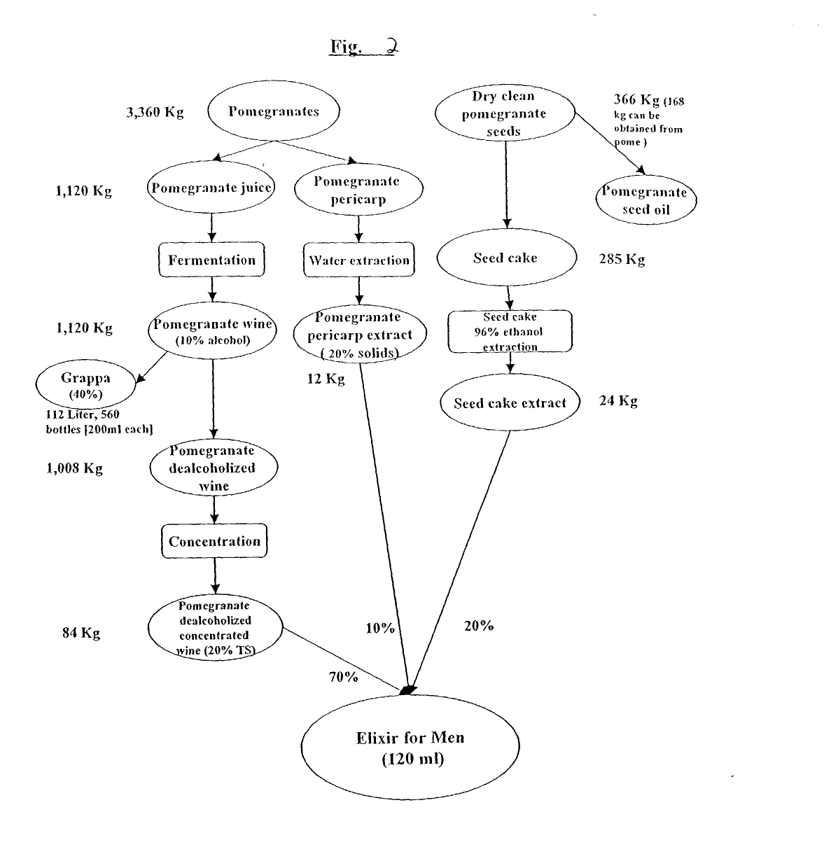

FIG. 2 shows production steps in manufacture of a pharmaceutical composition including 70% dealcoholized concentrated pomegranate wine, 10% aqueous extract of pomegranate pericarp, and 20% seed cake extract. The 120 ml of elixir represents 1000 doses of an elixir for men which could be delivered, for example, as gel-caps. The elixir is expected to have beneficial effects preventing benign prostatic hyperplasia (BPH) and/or prostate cancer. The production process is essentially as described for the elixir for women.

### Example 2

### Production of Gel-Caps from Pharmaceutical Compositions According to the Present Invention.

As detailed hereinabove, pharmaceutical compositions of the present invention may be provided in a wide variety of physical forms. One of these forms is gel-caps. Production of gelcaps typically includes the following steps:

1) Obtaining concentrated fermented juice and concentrated aqueous pericarp extracts.

2) Mixing the two components together, for example in a 9:1 ratio (fermented juice: pericarp).

3) Submitting these component extracts (together or individually) to supercritical fluid extraction using CO2 as a solvent with an ethanol modifier to obtain a polyphenol fraction. Suspending this polyphenol fraction in pomegranate seed oil in a ratio of, for example, 1:100 (polyphenol fraction: seed oil) to prepare the chemopreventive/phytoestrogen supplement. The prepared elixir may be encapsulated, for example in soft gel capsules.

### Example 3

### Pomegranate Oil and Juice Extract Synergistically Inhibit DU-145 Prostate Cancer Cell Proliferation

In order to demonstrate synergistic inhbition of cancer cell proliferation, the DU-145 cell line was employed. DU-145 is a highly invasive, poorly differentiated, androgen insensitive prostate cancer cell line, against which the individual pomegranate extracts have previously shown potent antiproliferative activity (Albrecht et al.(2002) “Pomegranate extracts potently inhibit growth, suppress invasion and modulate gene targets in prostate cancer cells.” Submitted for publication in Oncogene).

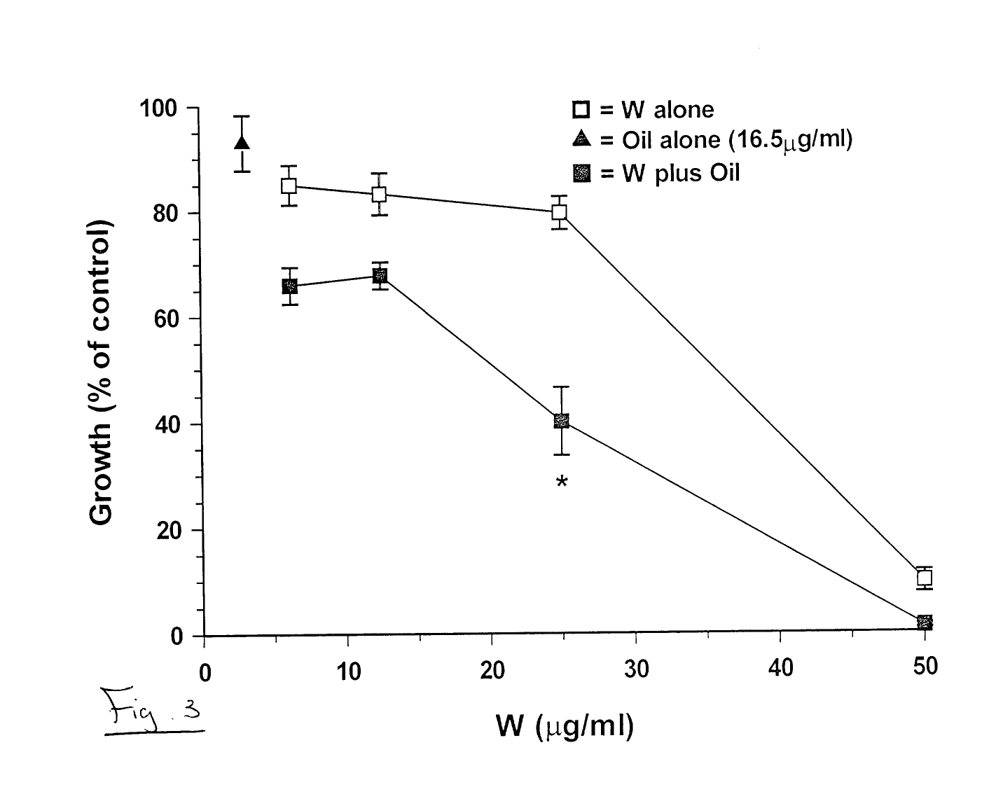

Results (summarized in FIG. 3) indicate that 16.5 μg/ml of pomegranate oil had only a slight effect on cell proliferation (87% of control growth ±7% (mean ±SEM). Varying amounts of an extract of fermented pomegranate juice rich in polyphenols (W) were employed either alone or in combination with the oil.polyphenols. The effect of the Oil/W combinations was compared to the sum of the effect of the standard dose of Oil and the dose of W used in the specific combination. Marked synergy was noted at several doses of W. For example,treatment with W at 25 μg/ml resulted in 76% ±4%, whereas the treatment combination resulted in 35% 9%. These results were significant at P≦0.05 (Student's t-test.). Thus, combining oil and W as a cancer treatment or prophylactic measure offers greater potential than either fraction alone.

### Example 4

### Pomegranate Oil and Juice Extract Synergistically Inhibit PC-3 Prostate Cancer Cell Invasion

In order to demonstrate synergistic inhbition of cancer cell invasion, the PC-3 cell line was employed. PC-3 is a moderately invasive, poorly differentiated, androgen insensitive prostate cancer cell line. PC-3 is known to penetrate an artificial basement membrane (Matrigel). Hepatocye growth factor (HPG) can be added to cultures to encourage this penetration and the extent of this invasion, or lack thereof, is used to predict ability of various compounds to prevent the spread of cancer.

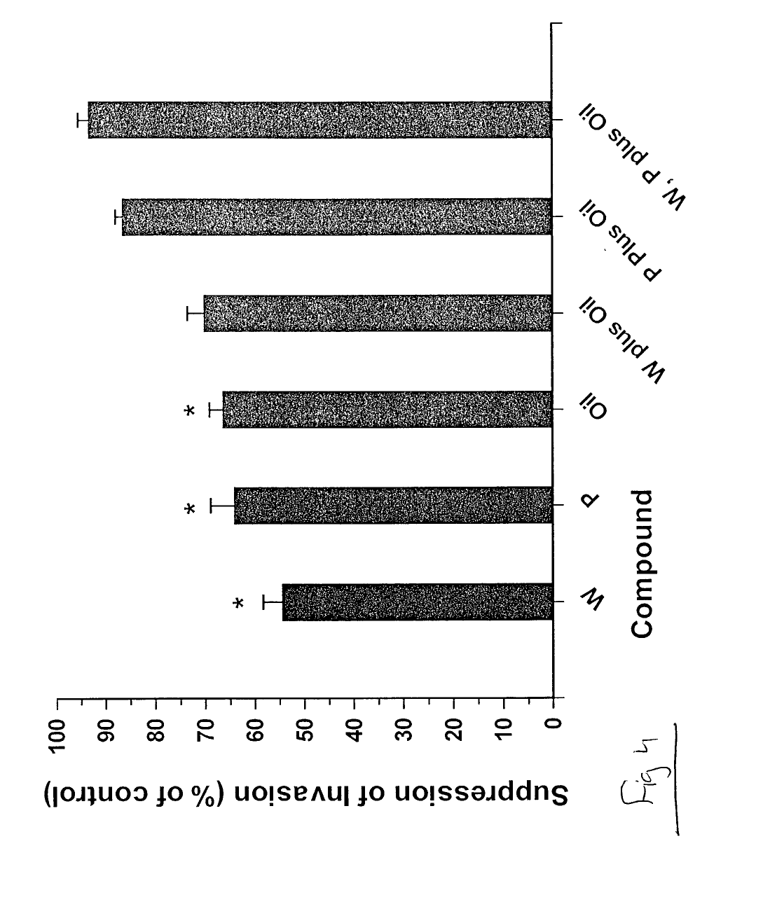

Pomegranate seed oil (Oil), an extract of fermented pomegranate juice rich in polyphenols (W) and an extract of pericarp rich in polyphenols (P) each individually inhibited invasion, relative to the hepatocyte growth factor/scatter factor positive control (Albrecht et al.(2002) “Pomegranate extracts potently inhibit growth, suppress invasion and modulate gene targets in prostate cancer cells.” Submitted for publication in Oncogene). When combined, such that the totals always equaled 3 μg/ml, synergism was observed (FIG. 4). Thus, 3 μg/ml of W alone resulted in 60±4% (±SEM) inhibition of the positive control, 3 μg/ml of P or Oil in 66±2% (p<0.01). The combination of 1.5 μg/ml P and 1.5 μg/ml Oil resulted in significantly greater inhibition, 85±1% (p<0.0001), and the combination of 1.0 μg/ml each of P, W and Oil produced 94±2% (p<0.01).

### EXAMPLE 5

### Pomegranate Pericarp and Juice Extracts Synergistically Inhibit DU-145 Prostate Cancer Cell Proliferation

The DU-145 cell line described hereinaabove (Example 3) was employed to demonstrate a synergistic anti-proliferative effect of pomegranate pericarp and juice extracts.

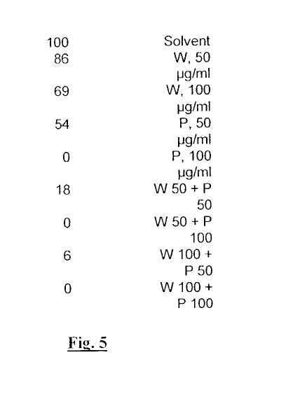

50 to 100 μg/ml of the same extract of fermented pomegranate juice described in Example 3 (W) were employed either alone or in combination with 50 to 100 μg/ml of pomegranate pericarp extract (P; Example 4) in the assay of Example 3. Results (summarized in FIG. 5) indicate that. 50 or 100 μg/ml (W) and 50 μg/ml (P) synergistically inhibited cell growth relative to W or P alone. These results were significant at P≦0.05 (Student's t-test.). Thus, combining P and W offer an additional potential cancer treatment or prophylactic measure.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A physiologically synergistic mixture of pomegranate extracts, the mixture comprising:
(a) a pomegranate juice extract; and
(b) pomegranate seed oil.

2. The mixture of claim 1, further comprising:
(c) a pomegranate pericarp extract.

3. The mixture of claim 1, wherein said juice extract includes an extract prepared using a solvent selected from the group consisting of water, an alcohol, ethyl acetate and carbon dioxide.

4. A physiologically synergistic mixture of pomegranate extracts, the mixture comprising:
(a) a pomegranate pericarp extract; and
(b) pomegranate seed oil.

5. The mixture of claim 4, further comprising:
(c) a pomegranate juice extract.

6. The mixture of claim 4, wherein said pericarp extract includes an extract prepared using a solvent selected from the group consisting of water, an alcohol, ethyl acetate and carbon dioxide.

7. A method of treating a cell proliferation disorder, the method comprising:
(a) preparing an extract of pomegranate pericarp;
(b) mixing said extract with a quantity of pomegranate seed oil to form a mixture;
(c) administering said mixture to a subject in a physiologically effective amount so that a progression of a cell proliferation disorder is retarded.

8. The method of claim 7, wherein said progression is retarded prior to appearance of clinical symptoms so that the method constitutes a prophylactic treatment for a cell proliferation disorder.

9. The method of claim 7, further comprising:
(d) preparing an additional extract of pomegranate juice and further including said additional extract in said mixture.

10. A method of treating a cell proliferation disorder, the method comprising:
(a) preparing an extract of pomegranate juice;
(b) mixing said extract with a quantity of pomegranate seed oil to form a mixture;
(c) administering said mixture to a subject in a physiologically effective amount so that a progression of a cell proliferation disorder is retarded.

11. The method of claim 10, wherein said progression is retarded prior to appearance of clinical symptoms so that the method constitutes a prophylactic treatment for a cell proliferation disorder.

12. The method of claim 10, further comprising:
(d) preparing an additional extract of pomegranate pericarp and further including said additional extract in said mixture.

13. A physiologically synergistic mixture of pomegranate extracts, the mixture comprising:
(a) a pomegranate pericarp extract; and
(b) a pomegranate juice extract.

14. The mixture of claim 13, wherein said pericarp extract includes an extract prepared using a solvent selected from the group consisting of water, an alcohol, ethyl acetate and carbon dioxide.

15. A method of treating a cell proliferation disorder, the method comprising:
(a) preparing a first extract of pomegranate pericarp;
(b) preparing a second extract of pomegranate juice;
(c) mixing said first and second extracts to form a mixture;
(d) administering said mixture to a subject in a physiologically effective amount so that a progression of a cell proliferation disorder is retarded.

16. The method of claim 15, wherein said progression is retarded prior to appearance of clinical symptoms so that the method constitutes a prophylactic treatment for a cell proliferation disorder.
